(19) United States
(12) Patent Application Publication
(10) Pub. No.: US 2008/0039943 A1
(43) Pub. Date: Feb. 14, 2008

(54) Set For Treating The Degeneracy Of An Intervertebral Disc

(76) Inventor: Regis Le Couedic, Andresy (FR)

Correspondence Address: Leffert, Jay & Polglaze, P.O.Box 581009, Minneapolis, MN, 55458-1009, US

(21) Appl. No.: 11/597,120

(22) PCT Filed: May 24, 2005
(86) PCT No.: PCT/FR05/01280

§ 371 Date: Nov. 21, 2006

(30) Foreign Application Priority Data
May 25, 2004 (FR) 0405611

(51) Int. Cl. A61F 2/44; A61B 17/56; C12N 5/00; A61B 17/58

(52) U.S. Cl. 623/17.16; 435/384; 606/100; 606/61; 606/73

(57) ABSTRACT

The invention concerns an assembly for treating degeneration of a damaged intervertebral disc arranged between two vertebrae. It comprises:
cells (22) which may or may not be analogous to those of the intervertebral disc and which can be implanted in said disc; and
at least one device (10) for stabilizing the two vertebrae, including:
two screws (12, 14), each screw being intended to be screwed into one of the vertebrae (V1, V2);
an elongate connection piece (16); and two fastening members (18, 20) for fastening the head of each of said screws to one of the ends of said connection piece, by which means it is possible to avoid excessive mechanical stresses being applied to the intervertebral disc.

The present invention relates to an assembly for treating degeneration of an intervertebral disc.

As is well known, the intervertebral disc is a means which joins vertebral bodies together and whose shape is that of a biconvex lens matching and fitting the articular surfaces of the vertebral bodies. They are made up of two parts: a peripheral part which is very hard and of a very compact texture, and a central part which is a soft and gelatinous substance.

For different reasons, diseases of the intervertebral disc may lead to greater or lesser degeneration of this disc, which is then no longer able to perform its normal function between the vertebrae.

Faced with this damage or degeneration of the intervertebral disc, it is necessary to employ a surgical technique to try to obtain regeneration of this disc and its normal biomechanical behaviour.

A technique aimed at obtaining this regeneration consists in implanting, in the damaged disc, cells intended to regenerate it, which cells may be of the same kind as those constituting the disc, or of another kind.

However, there may be some doubts as to the efficacy of this technique which would simply introduce new cells into a biomechanical environment that is already damaged, thus exposing them to excessive stresses, and thereby compromising their long-term survival. It seems, therefore, that such treatment has little chance of producing the expected result, that is to say regeneration of the intervertebral disc.

It is therefore important to define means for effectively obtaining the regeneration of the intervertebral disc in a lasting way. This is the object of the present invention.

The inventors have demonstrated the fact that, after implanting regeneration cells in the damaged intervertebral disc, the damage or necrosis of the cells constituting the disc did not disappear, or did not disappear sufficiently, on account of the mechanical pressure exerted on this disc by the vertebrae that surround it. Moreover, the inventors have also shown that some of the degeneration of the cells of the intervertebral disc could have a purely mechanical origin or that this purely mechanical origin could have created conditions favourable to the degeneration of this disc for other biological lesions.

The object of the invention is achieved by an assembly for treating degeneration of a damaged intervertebral disc arranged between two vertebrae, which assembly is characterized in that it comprises cells which may or may not be analogous to those of the intervertebral disc and which can be implanted in said disc; and at least one device for stabilizing the two vertebrae, including two screws, each screw being intended to be screwed into one of the vertebrae, an elongate connection piece, and two fastening members for fastening the head of each of said screws to one of the ends of said connection piece, by which means it is possible to avoid excessive mechanical stresses being applied to the intervertebral disc.

It will be appreciated that in the first instance the cells implanted in the intervertebral disc will permit regeneration of the cells of the intervertebral disc, and that this regeneration will be possible by virtue of the presence of the stabilizing device which is screwed into the vertebrae and thus maintains a spacing between these. It therefore limits the mechanical stresses applied to the intervertebral disc during treatment.

Moreover, as the device for stabilizing the vertebrae is screwed into these vertebrae, the invention can be used on patients who have undergone partial or total resection of the spinous processes of the vertebrae and whose spinous processes have too poor a bone quality (for example osteoporosis). It can also be used for the intervertebral disc between the fifth lumbar vertebra and the sacrum, since the first vertebra thereof does not generally have a spinous process. The invention can thus be used in cases where, for physiological reasons, stabilization between the processes cannot be effected.

The implantable cells used may be obtained by different methods such as collection from an intervertebral disc of the patient, collection of adult stem cells from the patient's bone marrow, or collection of embryonic stem cells.

In addition, the treatment assembly advantageously comprises means for injecting the cells into the disc, and these injection means can be of the type involving a syringe with cannula.

The connection piece of the device for stabilizing the vertebrae preferably has a certain flexibility.

It will thus be appreciated that stabilization of the vertebrae is not meant to be understood as strict relative immobilization of these two vertebrae, but maintaining the distance between these two vertebrae when the vertebral column is not flexed in the region concerned, while at the same time permitting certain limited flexion movements between these two vertebrae. This possibility of relative flexion movement causes a closing-together of the posterior parts of the two vertebrae opposite the screw zones of the stabilizing device. However this closing-together is limited, and it is temporary.

Other characteristics and advantages of the invention will become clearer from reading the following description of several embodiments of the invention which are given as non-limiting examples. In the description, reference is made to the attached figures, of which:

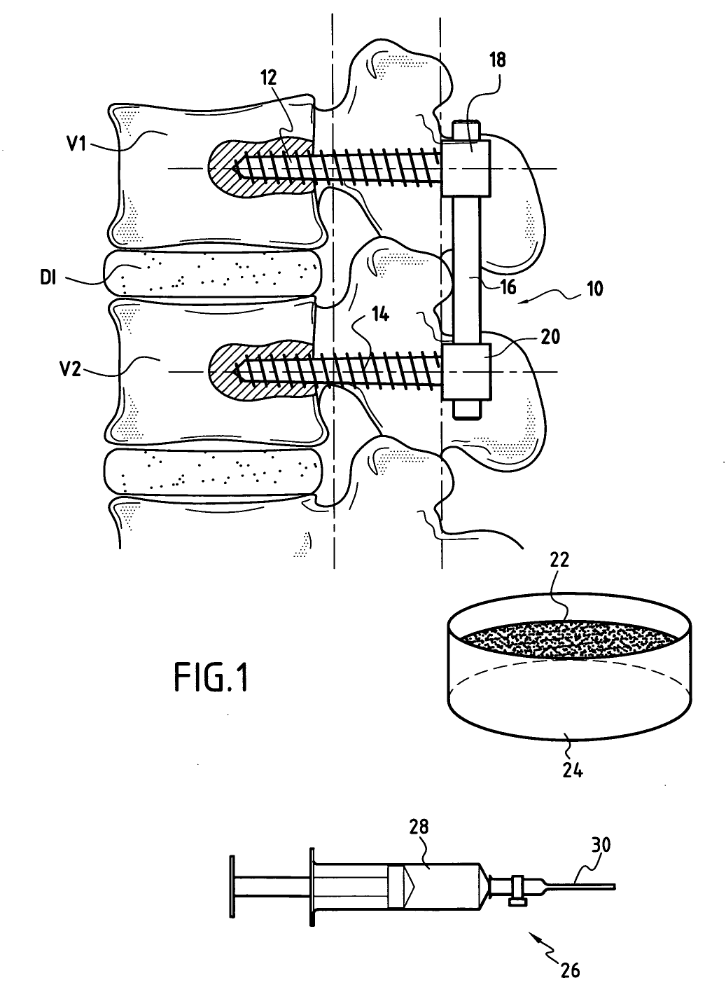

FIG. 1 shows the assembly according to the invention for treating degeneration of an intervertebral disc;

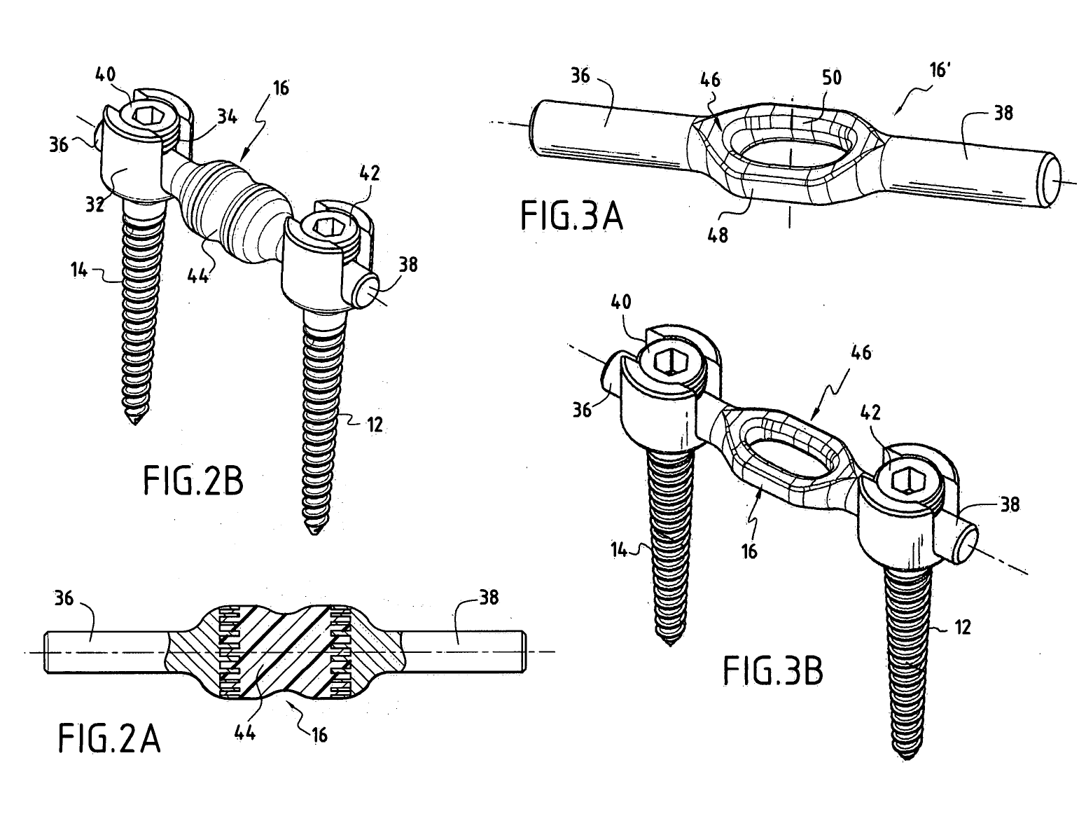

FIG. 2A is a cross-sectional view of a first embodiment of the connection piece of the immobilizing device;

FIG. 2B is a perspective view of the first embodiment of the immobilizing device;

FIG. 3A is a perspective view of a second embodiment of the connection piece of the immobilizing device;

FIG. 3B is a perspective view of the embodiment from FIG. 3A; and

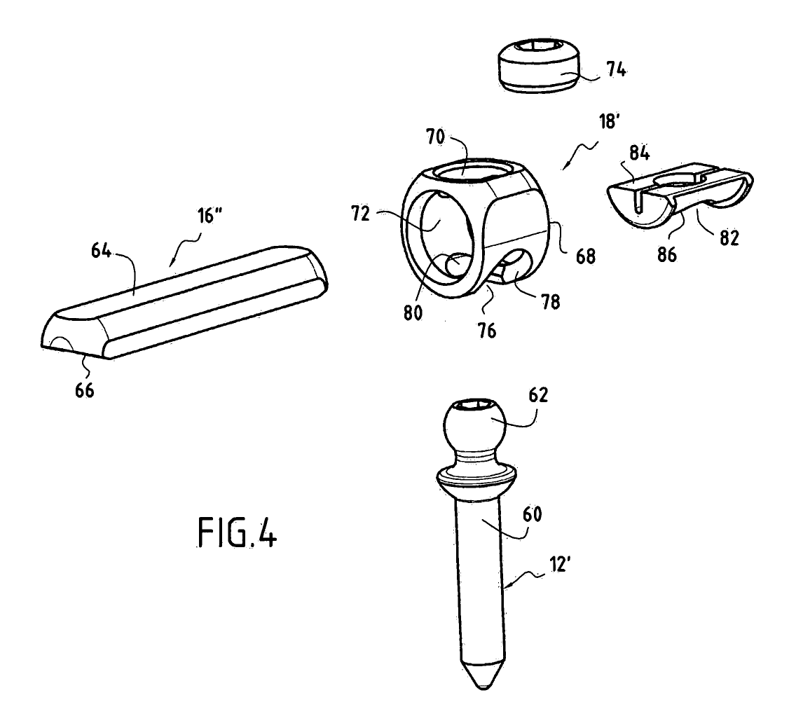

FIG. 4 is an exploded view of a third embodiment of the immobilizing device.

FIG. 1 first shows, in a simplified manner, a device 10 for stabilizing vertebrae, comprising two screws 12 and 14, a connection piece 16 and two fixation devices 18 and 20 for immobilizing the connection piece 16 relative to the screws 12 and 14. The figure also shows injectable cells for regenerating the intervertebral disc, these cells being symbolically designated by 22 and being shown in a culture dish 24. The means 26 for injecting the cells are also shown. In the particular embodiment of the invention shown in this figure, the injection means 26 are in the form of a syringe body 28 and a cannula 30.

As has already been explained, according to the invention the device 10 for stabilizing the vertebrae, which device constitutes part of the treatment assembly, is intended to make it possible to fix a given spacing between the vertebrae surrounding the intervertebral disc ID to be treated and to avoid excessive mechanical stresses being applied to this disc. Moreover, this treatment assembly includes injectable cells for therapy of the disc, the reflection or the implantation of the cells being able to take place at the same time as the stabilizing device is fitted, or after this, as will be explained below.

The devices for stabilization of vertebrae are themselves well known for their function of spacing apart and fixing the vertebrae. Such intervertebral immobilizing devices are described in particular in Patent Applications FR 02 12397, WO 02/07621 and WO 02/07622.

As has already been described in brief, the stabilizing device 10 is formed principally of two pedicle screws 12 and 14 which are screwed into the body of the two vertebrae that are to be secured. Each screw 12 and 14 is equipped with a fastening device 18 and 20 for fastening the heads of the screws to a connection piece 16 which thus defines the spacing between the two vertebrae. The connection piece 16 preferably has a certain flexibility.

Of course, in a complete system, two stabilizing devices are used, each device being mounted either side of the spinous processes of the vertebrae.

As regards the implantable cells that can be used for regenerating the intervertebral disc, they may be of different origins.

They may be cells collected from the patient's actual disc itself and cultured in a suitable medium (possibly in three dimensions) such as (alginate bead, porous PLA/TGA structure, collagen foam).

It is also possible to use autologous adult stem cells collected from the bone marrow, for example from the iliac crest, then placed in a culture medium identical to those mentioned above.

Finally, it is possible to use embryonic stem cells treated in the same way as has been indicated above.

These cells can be implanted in the intervertebral disc to be treated at the same time as the intervertebral implant is fitted, or they can be implanted in an entirely separate operation taking place before or after the intervertebral implant is fitted. In all cases, similar results are obtained.

In the case of a surgical intervention during which the implantation of the cells in the intervertebral disc and the fitting of the intervertebral implant take place simultaneously, the operating steps are as follows:
as regards the positioning of the patient on whom the operation is to be performed, he is placed lying on his front. A position of physiological lumbar lordosis is desirable.
the surgeon proceeds to expose the two vertebrae contiguous to the disc to be treated.

He puts the two stabilizing devices in place, using the connection pieces 16 to define a suitable spacing between the two vertebrae; and

he then proceeds to inject the culture cells into the intervertebral disc.

The preparations for the step of injecting the cells into the intervertebral disc depend on the type of culture medium which has been used. It may be necessary to separate them from these media before implanting them in the disc.

Referring now to FIGS. 2A and 2B, we will now describe a first embodiment of the device for stabilizing the vertebrae, which device is described in more detail in Patent Application No. WO 02/07622.

FIG. 2B shows the pedicle screws 12 and 14 and the connection piece 16 again. The pedicle screws each comprise a head 32 with a recess whose bottom is semi-cylindrical 34. The recess 34 in each screw head is intended to receive the cylindrical end 36, 38 of the connection piece 16. The ends 36 and 38 of the connection piece are immobilized with respect to the screws with the aid of locking screws 40 and 42 which cooperate with a female thread formed in the recesses 34 of the screw heads 32. In this embodiment of the stabilizing device, the central part 44 of the connection piece 16 is made of a material exhibiting a certain elasticity so as to permit limited movement between the vertebrae. This possibility improves patient comfort. However, as has already been explained, the relative movement of the two vertebrae is limited, thus avoiding application of excessive mechanical stresses to the intervertebral disc.

Referring to FIGS. 3A and 3B, we will now describe a second embodiment of the device for stabilizing the vertebrae, which device is described in more detail in Patent Application No. WO 02/07621. This device, when compared with the one described in connection with FIGS. 2A and 2B, differs only in terms of the design of the central part of the connection piece which is labelled 16′. In this case, the central part 46 of the connection piece is formed by two parallel arms 48 and 50 which are connected at their ends to the cylindrical end portions 36 and 38 of the connection piece. Thus, the central part 46 has, at its centre, a recess 50 which gives this central part a certain deformability while at the same time affording the same mechanical strength as the end portions 36 and 38.

Referring to FIG. 4, we will now describe a third embodiment of the device for stabilizing the vertebrae, which device is described in more detail in French Patent Application No. 02 12397.

In this embodiment, each pedicle screw, labelled 12′ in the figures, includes a threaded body 60 and a spherical fixation head 62. The connection piece, which is labelled 16″, is in the form of an elongate piece with a substantially flat upper face 64, and with a lower face 66 which is also substantially flat. The fixation device, which is labelled 18′, is formed in the first instance by a ring-shaped piece 68. The upper part of the ring 68 is provided with an orifice 70 which opens into the axial passage 72 of the ring. The orifice 70 is internally threaded to cooperate with a fixation screw 74. In its lower part, the ring additionally comprises a recess 76 with an insertion part 78 permitting passage of the spherical head 62 of the screw 12′, and a retention portion 80 which can cooperate with the lower part of the spherical head 62 of the screw in order to hold the latter in the spatial passage 72 of the ring 68. The fixation device 18′ finally comprises an intermediate piece 82 whose upper face 84 is flat and whose lower face 86 defines a spherical cap able to cooperate with the upper part of the spherical head 62 of the screw 12′.

After the pedicle screws 12′ have been screwed into the vertebrae at the appropriate sites, the fixation piece 68 is engaged on the spherical head 62 of the screw via its introduction recess 78 and is then pivoted about its axis in order to bring the spherical head 62 of the screw above the holding recess 80. The intermediate piece 82 and the end of the connection piece 16″ are then put in place in the axial passage 72. By screwing the screw 74 into the internally threaded orifice 70 of the ring, the connection piece 16″ is tightened and thus immobilized relative to the spherical head 62 of the pedicle screws 12′.

1-8. (canceled)

9. Assembly for treating degeneration of a damaged intervertebral disc arranged between two vertebrae, comprising:
cells which may or may not be analogous to those of the intervertebral disc and which can be implanted in said disc; and
at least one device for stabilizing the two vertebrae, including:
two screws, each screw being intended to be screwed into one of the vertebrae;
an elongate connection piece; and
two fastening members for fastening the head of each of said screws to one of the ends of said connection piece, by which means it is possible to avoid excessive mechanical stresses being applied to the intervertebral disc.

10. Treatment assembly according to claim 9, wherein said cells are such as are obtained by collecting a sample from an intervertebral disc of the patient and by culturing said collected cells.

11. Treatment assembly according to claim 9, wherein said cells are such as are obtained by collecting analogous adult stem cells from the patient's bone marrow and by culturing said cells.

12. Treatment assembly according to claim 9, wherein said cells are such as are obtained by collecting embryonic stem cells and by culturing said cells.

13. Treatment assembly according to claim 9, further comprising means for injecting said implantable cells into the intervertebral disc of the patient.

14. Treatment assembly according to claim 9, wherein said connection piece of the stabilizing device has on the whole a certain flexibility.

15. Treatment assembly according to claim 14, wherein said connection piece has two ends made of a first material, and a central part made of a second material having a coefficient of elasticity superior to that of the first material.

16. Treatment assembly according to claim 14, wherein said connection piece is made of a single material, and in that the two ends of said connection piece are solid and in that its central part has a recess extending in the direction of the length of said piece.
